(19) United States
(12) Patent Application Publication
(10) Pub. No.: US 2009/0018493 A1
(43) Pub. Date: Jan. 15, 2009

(54) Implantable catheter assembly

(76) Inventors: Stephen R. Ash, Lafayette, IN (US); Kenneth E. Brown, West Layfayette, IN (US)

Correspondence Address: KRIEG DEVAULT LLP, ONE INDIANA SQUARE, SUITE 2800, INDIANAPOLIS, IN, 46204-2079, US

(21) Appl. No.: 11/827,025

(22) Filed: Jul. 10, 2007

(51) Int. Cl. A61M 1/28; B65D 69/00

(52) U.S. Cl. 604/29; 206/571

(57) ABSTRACT

Assemblies, kits and methods are provided for catheter assemblies that include a coupling member with first and second access ports to which first and second intrabody members are coupled. The coupling member defines an internal chamber which is in communication with lumens of the first and second intrabody members. In an implantation orientation each of the first and second intrabody members is in a straightened configuration relative to the coupling member and in an implanted orientation each of the first and second intrabody members is in a contracted configuration relative to the coupling member.

The present application relates to an implantable catheter assembly and systems, manners and kits for using the same. In one form, but not exclusively, the present application relates to an assembly for use in peritoneal dialysis.

The use of implantable catheters to deliver and/or drain one or more fluids from the body of a patient has become commonplace. However, current implantable catheter assemblies may be subject to certain complications which can make the use of these implantable catheters inefficient, and in some cases, completely inoperative. For example, certain catheter assemblies may be susceptible to occlusion by one or more materials, such as for example, blood or a blood product which has coagulated therein. In another example, current catheter assemblies may not effectively drain the one or more fluids from the body as desired. One such occurrence may be, for example, the incomplete drainage of a spent dialysate infused into the peritoneal cavity of a patient during peritoneal dialysis. Thus, there is a need for additional contributions in this area of technology.

One form of the present application is a unique implantable catheter assembly. Other forms include unique methods, systems, devices, instrumentation, and apparatus involving an implantable catheter assembly.

According to one aspect, there is provided a catheter assembly for use in peritoneal dialysis. The catheter assembly includes a first intraperitoneal member including a first coiled portion at a distal end thereof and defining a first lumen extending from a proximal end toward the distal end. The catheter assembly also includes a second intraperitoneal member including a second coiled portion at a distal end thereof and defining a second lumen extending from a proximal end toward the distal end. The proximal end of the first intraperitoneal member extends from a coupling member at a first access port and the proximal end of the second intraperitoneal member extends from the coupling member at a second access port. The catheter assembly further includes a transabdominal member extending from the coupling member at a third access port and defining a third lumen in communication with the first and second lumens.

Another aspect includes a catheter assembly including a resilient, pliable coupling member defining an internal chamber and including a first portion with a first access port and a second access port. The coupling member further includes a second portion defining a third access port. Each of the first, second and third access ports is in communication with the internal chamber defined by the coupling member. First and second intrabody members are further included and respectively extend from the first and second access ports and define lumens in communication with the internal chamber. The catheter assembly is configurable to an implantation orientation with each of the first and second intrabody members in a straightened configuration and an implanted orientation with each of the first and second intrabody members is in a contracted configuration.

In yet another aspect there is provided a catheter assembly including an elongated main member including a coupling portion disposed at a distal end thereof. The catheter assembly also includes a first intrabody member including a proximal portion and a distal portion defining a first coiled section and a second intrabody member including a proximal portion and a distal portion defining a second coiled section. The proximal portions of the first and second intrabody members extend from the coupling member along a first longitudinal axis and the first and second coiled sections are positioned on substantially opposite sides of the first longitudinal axis.

In still another aspect, there is provided a flow-through system for performing peritoneal dialysis. The system includes an intraperitoneal supply member structured to infuse a dialysate into the peritoneal cavity. The system also includes an intraperitoneal drain member which is spaced from the supply member. The drain member includes a first collecting member including a first coiled section at a distal end thereof and a second collecting member including a second coiled section at a distal end thereof. Each of the collecting members is further structured to remove the dialysate from the peritoneal cavity.

In an even further aspect there is provided a method for positioning a catheter assembly for peritoneal dialysis. The method includes providing a catheter assembly including a coupling member which includes a transabdominal portion. The assembly also includes first and second intraperitoneal members which extend from the coupling member and include distal coiled sections. The method also includes providing a pair of stylets configured to pass through the coupling member and into communication with lumens of the first and second intraperitoneal members; inserting a first one of the stylets into the lumen of the first intraperitoneal member and thereby substantially straightening the distal coiled section thereof; inserting a second one of the stylets into the lumen of the second intraperitoneal member and thereby substantially straightening the distal coiled section thereof; implanting the catheter assembly at a prepared surgical location with the coiled sections substantially straightened; and removing the first and second stylets from the lumens of the intraperitoneal members and thereby reforming the distal coiled sections to an unstraightened configuration.

In a yet a further aspect, a kit for implanting a catheter assembly is provided. The kit includes a catheter assembly including a main member and first and second intrabody members extending from a coupling member. Each of the first and second intrabody members further includes a coiled portion positioned at a distal end thereof. The kit further includes first and second stylets with each including an elongated shaft portion and an enlarged head portion at a distal end of the shaft portion. Packaging for holding the catheter assembly and the first and second stylets in a sterilized condition until implantation of the catheter assembly is also provided.

Further embodiments, forms, features and aspects of the present application shall become apparent from the detailed description and figures provided herewith.

### BRIEF DESCRIPTION OF THE DRAWINGS

FIG. 1 is a plan view of an implantable catheter assembly.

FIG. 2 is a plan view of the implantable catheter assembly of FIG. 1 in an implantation orientation.

FIG. 3 is a right side view of the implantable catheter assembly of FIG. 1.

FIG. 4 is an enlarged plan view of one embodiment of a coupling member of the implantable catheter assembly of FIG. 1.

FIG. 5 is a section view of a pair of intrabody members taken along view line 5-5 in FIG. 2.

FIG. 6 is an enlarged partial section view of a portion of the intrabody members illustrated in FIG. 2.

FIG. 7 is a schematic diagram illustrating one example of a system with which the implantable catheter assembly of FIG. 1 may be used.

FIG. 8 is a schematic diagram illustrating another example of a system with which the catheter assembly of FIG. 1 may be used.

FIG. 9 is an enlarged partial sectional view of the coupling member of an alternative embodiment implantable catheter assembly.

FIG. 10 is a plan view of the implantable catheter assembly of FIG. 2 including a pair of stylets inserted into the lumens of the catheter assembly.

FIG. 11 is a plan view of the stylets illustrated in FIG. 10.

FIG. 12 is a section view of the intrabody members of FIG. 10 positioned relative to a retractor and including stylets positioned in the lumens thereof.

FIG. 13 is a plan view of a surgical kit including the catheter assembly of FIG. 1 and the stylets of FIG. 11.

### DETAILED DESCRIPTION OF PREFERRED EMBODIMENTS

For the purposes of promoting an understanding of the principles described herein, reference will now be made to the embodiments illustrated herein and specific language will be used to describe the same. It will nevertheless be understood that no limitation of the scope of any subject matter described and claimed herein is thereby intended. Any alterations and further modifications in the described processes, systems, or devices, and any further applications of the principles described and illustrated herein, are contemplated as would normally occur to one skilled in the art.

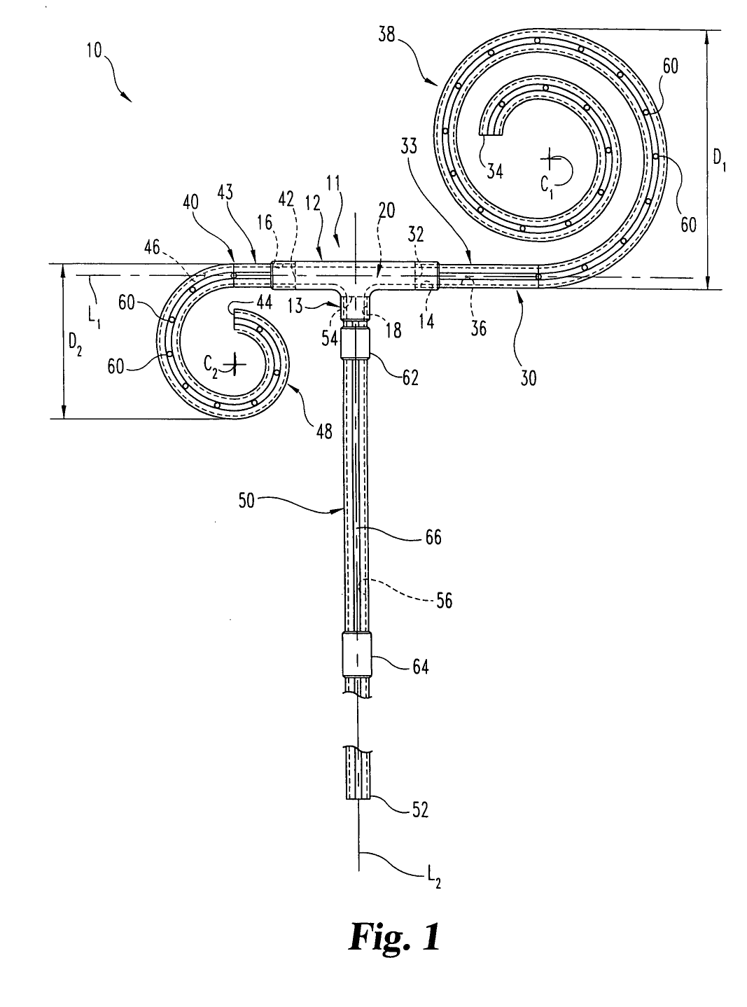

With reference to FIG. 1, there is illustrated in plan view an implantable catheter assembly 10 according to one embodiment of the present application. Catheter assembly 10 may be implanted at one or more regions of a mammalian patient to supply and/or drain one or more fluids from the patient. For example, in one non-limiting form, catheter assembly 10 may be implanted at a wound site to drain one or more bodily fluids collecting therearound. These fluids may include blood, plasma, plasma water, lymph, and purulent exudate. In another form, catheter assembly 10 may be implanted to deliver one or more fluids to the patient. These fluids may include, for example, an antibiotic, an antimicrobial agent, an analgesic, saline, nutritional fluids and chemotherapy agents.

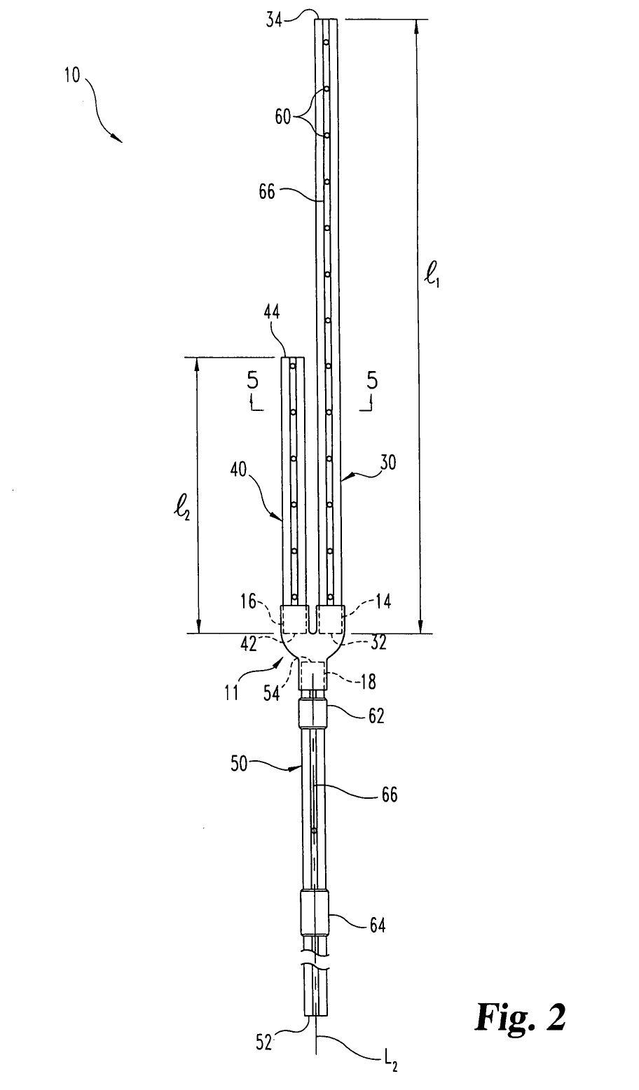

In one contemplated application, catheter assembly 10 may be used to supply a dialysate into the peritoneal cavity of the patient. In another exemplary application, catheter assembly 10 may be used to drain a spent dialysate from the peritoneal cavity of the patient. It should be further appreciated that catheter assembly 10 may be suitable for use in a variety of applications in which current catheter assemblies are used. Moreover, catheter assembly 10 may be manufactured from a variety of suitable materials, including for example, but not limited to, silicone or polyurethane. Accordingly, one or more of the components of catheter assembly 10 may be resiliently pliable to facilitate positioning of the catheter assembly between an implanted orientation illustrated in FIG. 1 and an implantation and/or withdrawal orientation illustrated in FIG. 2. Aspects regarding the implanted and implantation orientations are set forth in further detail below. Additionally, as should be apparent in view of the following description, the implanted and implantation orientations of FIGS. 1 and 2 are illustrative only, and each orientation may vary as the configuration of the catheter assembly varies and/or in relation to the anatomical features at an implantation location. Moreover, a person skilled in the relevant field will appreciate that, in use, various external forces will be exerted on catheter assembly 10, and due to the pliable nature of the materials from which catheter assembly 10 is made, the external forces will generally cause deformation of the catheter assembly 10 from the shapes described and illustrated herein. As such, the features described herein should be taken to describe the configuration of catheter assembly 10 in the absence of external forces and, due to the resilient nature of the materials, the configuration to which catheter assembly 10 returns when external forces are removed.

Catheter assembly 10 generally includes a coupling member 11, a first intrabody member 30, a second intrabody member 40 and a main member 50. While each of the members 11, 30, 40 and 50 are illustrated as unitary pieces, it should be appreciated that one or more of the members 11, 30, 40 and 50 can be made up of one or more pieces that are coupled together. Moreover, in an alternative embodiment, one or more of members 11, 30, 40 and 50 can be integral with one another. For example, and without limitation, members 11, 30 and 40 can be integrally formed, thereby comprising a single unitary piece.

As used herein, the term “coupling member” generally refers to a member that includes at least one internal chamber with which one or more of the lumens of the members 30, 40 and 50 communicate. In the illustrated embodiment, coupling member 11 includes a first portion 12 and a second portion 13. First portion 12 extends generally along a longitudinal axis L₁ and defines a first access port 14 and a second access port 16, both of which are in communication with an internal chamber 20 defined by coupling member 11. Second portion 13 generally extends along a second longitudinal axis L₂ and defines a third access port 18 which is also in communication with the internal chamber 20. As used herein, the term “access port” generally refers to a location where lumens of the members 30, 40 and 50 communicate with internal chamber 20. Moreover, while longitudinal axes L₁, L₂ are illustrated as extending substantially perpendicular to each other, it is contemplated that coupling member 11 can be alternatively shaped. For example, the longitudinal axis L₂ can extend obliquely relative to the longitudinal axis L₁ and in the direction of either first access port 14 or second access port 16. Moreover, members 30, 40 and 50 can lie in substantially the same plane or can lie in different planes. For example, in one embodiment, members 30 and 40 lie in one plane and member 50 lies in a different plane. Further details regarding access ports 14, 16 and 18 as well as internal chamber 20 are set forth below with respect to FIG. 4.

As illustrated in FIGS. 1 and 2 for example, main member 50 generally extends along longitudinal axis L₂ forming an axial extension of second portion 13 of coupling member 11. However, it should be appreciated that the position of main member 50 can be varied either singly or in combination with an adjustment of second portion 13 along the longitudinal axis L₂. Main member 50 generally defines a lumen 56 which opens at and extends between a proximal end 52 and a distal end 54. Distal end 54 is coupled with coupling member 11 at third access port 18 with lumen 56 in communication with internal chamber 20 of coupling member 11. Main member 50 may generally have a length between ends 52, 54 sized to extend from a desired intrabody position of the coupling member 11 to a position at or slightly outside the skin of a patient. In another form, main member 50 may have a length sized to extend beyond the skin of a patient for coupling with a supply source or a drain. In still another embodiment, it is contemplated that the main member 50 may be coupled with another intrabody member before it reaches the skin of a patient. In one specific embodiment, the length of main member 50 is in the range from about 20 to about 40 cm. In another specific embodiment, the length of main member 50 is in the range from about 25 to about 35 cm. In yet another specific embodiment, the length of main member 50 is about 30 cm.

The proximal end 52 of main member 50 can be appropriately configured to facilitate coupling thereof with an external component. For example, proximal end 52 can include a male or female portion which is structured to releasably couple with a corresponding female or male portion of the external component, such as a supply member, drainage tube or reservoir. Additionally, it should be appreciated that proximal end 52 can include one or more features structured to seal lumen 56 when catheter assembly 10 is not desired to drain or supply fluid.

In the illustrated embodiment, main member 50 includes a first cuff 62 positioned adjacent to coupling member 11 and a second cuff 64 spaced apart from first cuff 62 toward proximal end 52. The cuffs 62, 64 can be selected from a variety of commercially available products as would be appreciated by those having skill in the art. In one specific embodiment, it is contemplated that cuffs 62, 64 can be Dacron® or polyester felt cuffs. In this embodiment, first cuff 62 can be structured to promote tissue ingrowth at an intrabody position of the patient while second cuff 64 promotes tissue ingrowth at a position spaced apart from the intrabody position. In one non-limiting example, the main member 50 can be sized and structured to extend through the abdominal wall with coupling member 11 implanted in the peritoneal cavity of a patient and proximal end 52 located at a position outside the skin of the patient. In this embodiment, first cuff 62 is positioned to promote tissue ingrowth within or just outside the rectus muscle of the patient while second cuff 64 is positioned to promote tissue ingrowth in the subcutaneous space just below the skin of the patient and near the exit site. It should be appreciated that tissue ingrowth facilitated by the cuffs 62, 64, amongst other functions, secures placement of catheter assembly 10, seals the implantation site and prevents infection at the catheter site.

In the illustrated embodiment, main member 50 also includes a radiopaque marker 66 which also extends along the first and second intrabody members 30, 40. It should be appreciated that radiopaque marker 66 can be of any known form, including, for example, an iodine-based marker or barium sulfate. The maker 66 can be positioned relative to certain structural features of catheter assembly 10 in order to facilitate radiographic viewing of catheter assembly 10 relative to various anatomical features of the patient. Moreover, while only one marker 66 has been positioned on catheter assembly 10, it is contemplated that more than one marker 66 can be positioned on catheter assembly 10 to better facilitate implantation and positioning of catheter assembly 10 in various applications. In other embodiments, marker 66 is omitted.

First intrabody member 30 is coupled with coupling member 11 at the first access port 14 while second intrabody member 40 is disposed oppositely of the first intrabody member 30 and is coupled with coupling member 11 at second access port 16. As used herein, the term “intrabody member” refers to a portion of catheter assembly 10 which will be either substantially or completely positioned in the body of a patient when in use. For example, in one embodiment, intrabody members 30, 40 are structured for insertion into the peritoneal cavity of a patient, in which case intrabody members 30, 40 are referred to as “intraperitoneal members.”

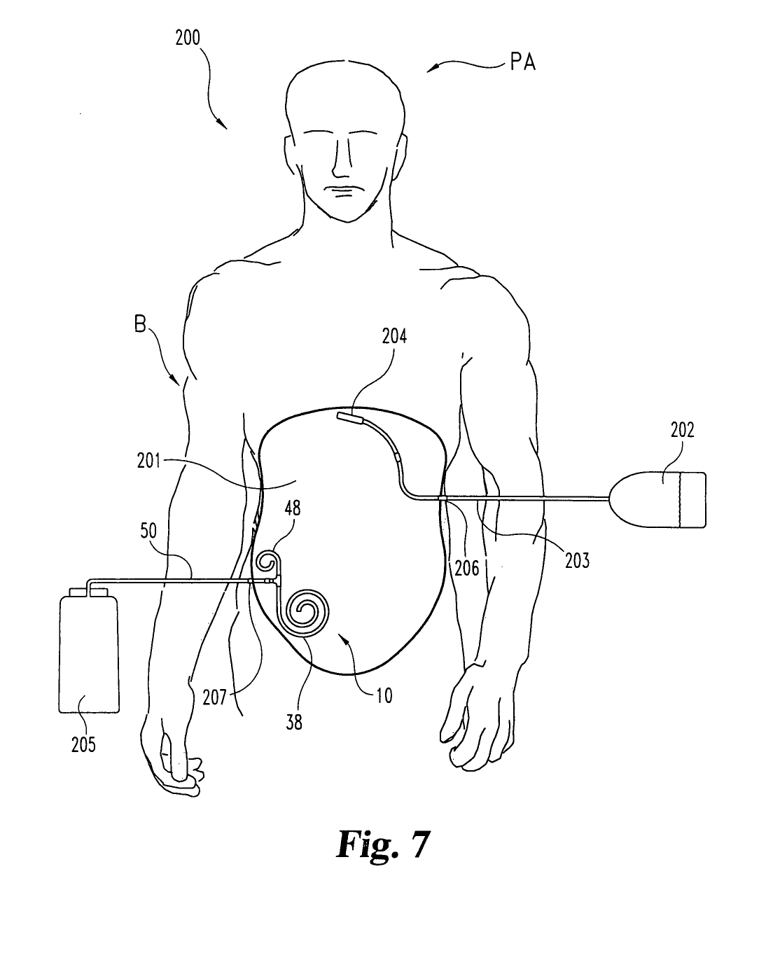

First intrabody member 30 includes a proximal portion 33 with a proximal end 32 positioned adjacent internal chamber 20 in engagement with coupling member 11. First intrabody member 30 also includes a distal end 34 opposite proximal end 32. A lumen 36 extends between proximal end 32 and distal end 34 in fluid communication with internal chamber 20. Moreover, first intrabody member 30 is provided with a plurality of openings 60 structured to pass a fluid into and/or from lumen 36. Further details regarding openings 60 are set forth below with respect to FIG. 7. First intrabody member 30 also includes a coiled portion or section 38 disposed near distal end 34. As illustrated in FIG. 1, coiled section 38 generally coils around the center C₁, and distal end 34 is positioned adjacent to the center C₁ of the coiled section 38. Additionally, coiled section 38 generally includes a dimension D₁ corresponding to the diameter across the largest portion of coiled section 38.

As indicated above, second intrabody member 40 is positioned opposite the first portion 12 of coupling member 11 from first intrabody member 30. Similar to first intrabody member 30, second intrabody member 40 includes a proximal portion 43 including a proximal end 42 which is positioned adjacent the internal chamber 20 in engagement with the coupling member 11. Intrabody member 40 also includes a distal end 44 opposite proximal end 42. A lumen 46 extends between proximal end 42 and distal end 44 in fluid communication with internal chamber 20. Similar to first intrabody member 30, second intrabody member 40 is also provided with a plurality of openings 60 structured to pass a fluid into and/or from lumen 46. Second intrabody member 40 also includes a coiled portion or section 48 disposed near distal end 44. As illustrated in FIG. 1, coiled section 48 generally coils around the center C₂, and distal end 44 is positioned adjacent to the center C₂ of the coiled section 48. Additionally, coiled section 48 generally includes a dimension D₂ corresponding to the diameter across the largest portion of coiled section 48. In the embodiment depicted in FIG. 1, coiled section 48 is more tightly coiled than coiled section 38, and dimension D₂ of coiled section 48 is less than dimension D₁ of coiled section 38. In alternative embodiments, coiled sections 48 and 38 can have dimensions corresponding to one another, or coiled section 38 can be more tightly coiled than coiled section 48.

As most clearly seen when catheter assembly 10 is in the implantation orientation of FIG. 2, first intrabody member 30 also includes a length l₁ corresponding to the distance between the proximal end 32 and the distal end 34 and second intrabody member 40 includes a length l₂ which extends between proximal end 42 and distal end 44, and the length l₁ is greater than the length l₂. In one specific embodiment, the length l₁ is in the range from about 15 to about 35 cm and the length l₂ is in the range from about 4 to about 12 cm. In another specific embodiment, length l₁ is in the range from about 20 to about 30 cm and the length l₂ is in the range from about 6 to about 10 cm. In yet another specific embodiment, the length l₁ is in the range from about 23 to about 25 cm and the length l₂ is in the range from about 7 to about 9 cm.

Each of the proximal portions 33, 43 generally extends along the longitudinal axis L₁ and forms axial extensions of first portion 12. Coiled section 38 is positioned on a substantially opposite side of longitudinal axis L₁ as coiled section 48. In the illustrated embodiment, coiled section 48 is positioned proximally of and on the same side of longitudinal axis L₁ as main member 50.

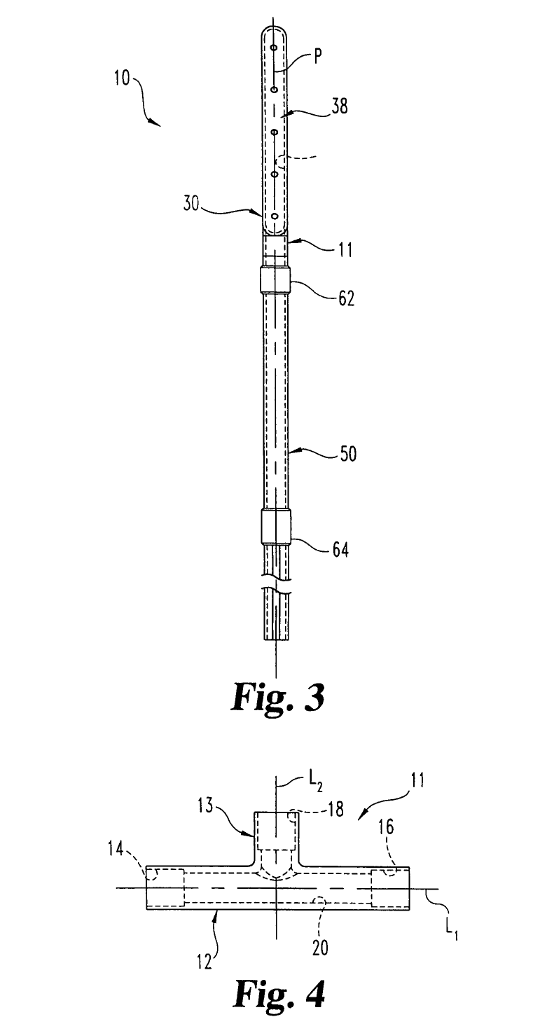

However, it should be appreciated that that the positions of coiled sections 38, 48 can be altered relative to longitudinal axis L₁. For example, in one alternative embodiment, coiled section 38 is positioned on the same side of longitudinal axis L₁ as main member 50. Moreover, further variations of the positioning of coiled sections 38, 48 relative to main member 50 are contemplated. For example, as illustrated in FIG. 3, intrabody members 30, 40, coupling member 11 and main member 50 are positioned substantially in a common plane P. However, in an alternative embodiment, it is contemplated that all or part of one or both of the intrabody members 30, 40 can be positioned in a plane extending obliquely or transversely to the plane P while main member 50 lies in plane P.

While coiled sections 38, 48 have several similar features and characteristics, certain aspects of coiled sections 38, 48 are different. For example, as stated above, coiled section 48 is generally more tightly coiled around the center C₂ than coiled section 38 is around the center C₁. In view of the foregoing described differences between the curvature of the respective coils and the lengths l₁, l₂, it should be appreciated that the dimension D₁ corresponding to the diameter across the largest portion of coiled section 38 is greater than the dimension D₂ corresponding to the diameter across the largest portion of coiled section 48, a feature which is also illustrated in FIG. 1. In an alternative non-illustrated embodiment, it is contemplated that length l₂ can be greater than length l₁ and that dimension D₁ corresponding to the diameter across the largest portion of coiled section 38 is less than the dimension D₂ corresponding to the diameter across the largest portion of coiled section 48.

With respect to FIG. 4, there is illustrated in further detail coupling member 11. As indicated above, coupling member 11 includes an internal chamber 20 which is in communication with access ports 14, 16 and 18. The access ports 14, 16 and 18 can be in the form of a socket into which the ends 32, 42 and 54 of the members 30, 40 and 50 can be inserted, respectively. In this embodiment, access ports 14, 16 and 18 can include an internal diameter which is slightly smaller than the external diameter of the respective members 30, 40 and 50 to provide an interference fit therebetween. Moreover, as one having skill in the art would appreciate, one or more adhesives can be applied to one or more of the access ports 14, 16 and 18 and the members 30, 40 and 50 to enhance coupling therebetween. Still, in another non-illustrated embodiment, it is contemplated that one or more of the members 30, 40 and 50 can be integrally formed with coupling member 11 or can be coupled with coupling member 11 via a threaded interconnection or other engagement configuration. In each of these various embodiments, it should be appreciated that each of the lumens 36, 46, 56 is in fluid communication with internal chamber 20. Accordingly, a fluid can be distributed through internal chamber 20 to and/or from any of lumens 36, 46 and 56.

FIG. 5 is a sectional view of intrabody members 30, 40 taken along view line 5-5 in FIG. 2. Each of the intrabody members 30, 40 includes exterior wall portions 31, 41, respectively, which surround and define the lumens 36, 46. As illustrated with respect to intrabody member 40, lumen 46 includes an inner diameter d₁ extending between the exterior wall portion 41. The inner diameter d₁ is generally sized to facilitate unobstructed passage of a fluid through lumen 46. In one specific embodiment, diameter d₁ is in the range from about 3 to about 5 mm. In another specific embodiment, it is contemplated that inner diameter d₁ is about 4 mm. Additionally, an outer diameter d₂ extends across wall portion 41 of the intrabody member 40. In one specific embodiment, outer diameter d₂ is in the range from about 0.25 to about 1.25 mm. In another specific embodiment, the outer diameter d₂ is in the range from about 0.5 to about 1.0 mm. In yet another specific embodiment, the outer diameter d₂ is in the range from about 0.6 to about 0.9 mm. In even another specific embodiment, the outer diameter d₂ is about 0.75 mm. While the inner diameter d₁ and the outer diameter d₂ have been described with respect to intrabody member 40, it should be understood that intrabody member 30 can also include inner and outer diameter dimensions similar to those described with respect to intrabody member 40. Moreover, main member 50 can also include inner and outer diameter dimensions similar to those of intrabody members 30, 40.

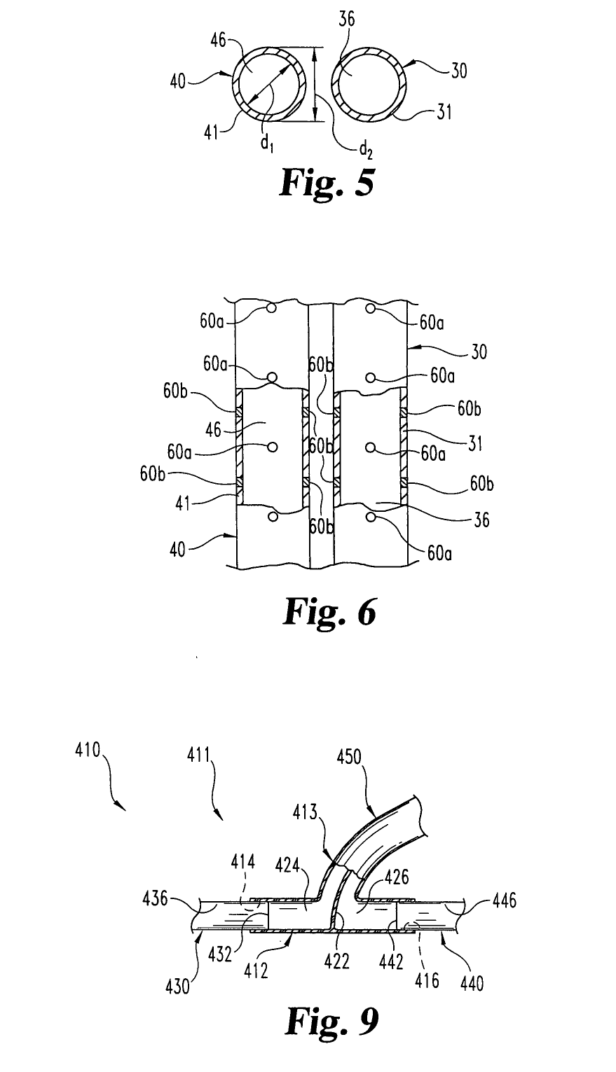

Further details regarding first and second intrabody members 30, 40 are shown in an enlarged partial section view in FIG. 6. More particularly, FIG. 6 depicts the relative positioning of the openings 60. Openings 60 are generally provided in pairs which are spaced apart from one another about 180° along the exterior walls 31, 41. For example, each of the openings 60a is positioned about 180° and opposite from another of the openings 60a along the exterior wall 31, 41 while each of the openings 60b is positioned about 180° and opposite from another of the openings 60b along the exterior wall 31, 41. Additionally, each of the opposite pairs of openings 60a is spaced longitudinally apart from and is angularly offset about 90° around the circumference of exterior walls 31, 41 from an adjacent pair of openings 60b. It should be appreciated that the size and/or positioning of the openings 60a, 60b can be varied in alternative embodiments. For example, the pairs of openings 60a, 60b can be more or less closely spaced to increase or decrease the total number of openings 60, the size of the openings 60 can be enlarged or reduced in order to increase or restrict fluid flow through the openings 60 and other spacing patterns and angular offsets between adjacent openings are contemplated.

Function of catheter assembly 10 will now be further discussed. For example, in one procedure, main member 50 can be coupled with a fluid source. The source can provide a fluid which passes through the lumen 56 of main member 50 and into internal chamber 20 of coupling member 11, which in turn disperses the fluid into lumens 36, 46 of intrabody members 30,40, respectively. Openings 60 can then distribute the fluid from lumens 36, 46 into the patient. In another procedure, openings 60 collect a fluid from an intrabody space and direct it into lumens 36, 46 of intrabody members 30, 40, respectively. The fluid is then passed from lumens 36, 46 into internal chamber 20 where it is directed into lumen 56 of main member 50 and drained from the patient. It should be appreciated that catheter assembly 10 can be asynchronously used to supply and drain a fluid from the patient. For example, the assembly 10 can be used for Continuous Ambulatory Peritoneal Dialysis (CAPD), Automated Peritoneal Dialysis (APD), or Continuous Cycling Peritoneal Dialysis (CCPD), just to name a few examples, where catheter assembly 10 provides a dialysate to the peritoneal cavity of the patient and subsequently removes the dialysate from the peritoneal cavity of the patient after a desired period of time.

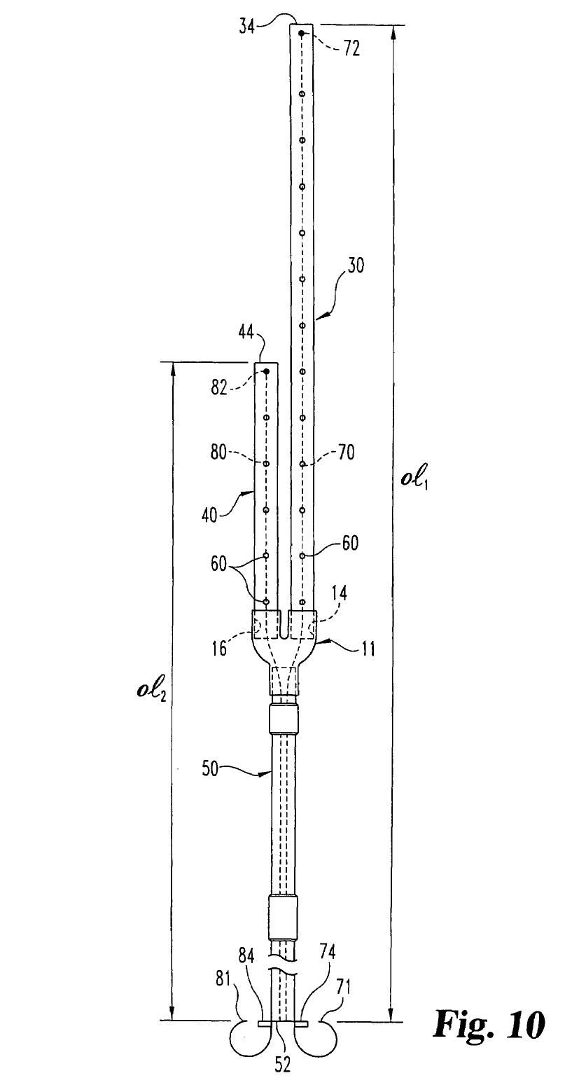

Also contemplated by the present application are methods for inserting the catheter assemblies into the patient. It should be appreciated that the described methods can be employed with standard catheter placement systems, including for example, the Quill® and Y-TEC® assemblies. As indicated above, FIG. 1 generally illustrates catheter assembly 10 in an implanted orientation. Specifically, in this form, intrabody members 30, 40 are generally contracted relative to the coupling member 11. It should also be appreciated that this orientation may be typical for catheter assembly 10 before implantation as well. However, to facilitate implantation of catheter assembly 10, each of the intrabody members 30, 40 is uncoiled and becomes straightened relative to the coupling member 11 as illustrated in FIGS. 2 and 10. It should be noted that lumens 36, 46 and 56 have not been illustrated in phantom in each of FIGS. 2 and 10 in order to provide clarity with respect to the implantation orientation.

To facilitate transition of catheter assembly 10 to the implantation orientation, coupling member 11 is generally folded to position the first and second access ports 14, 16 adjacent to one another along longitudinal axis L₂. A first stylet 70 is inserted into main member 50 and through coupling member 11 into the lumen of intrabody member 30. As stylet 70 is moved distally toward distal end 34, coiled section 38 becomes uncoiled and intrabody member 30 becomes substantially linear as illustrated in FIGS. 2 and 10. Similarly, a second stylet 80 is inserted into main member 50 and through coupling member 11 into the lumen of intrabody member 40. As stylet 80 is moved distally toward distal end 44, coiled section 48 becomes uncoiled and intrabody member 40 becomes substantially linear as illustrated in FIGS. 2 and 10.

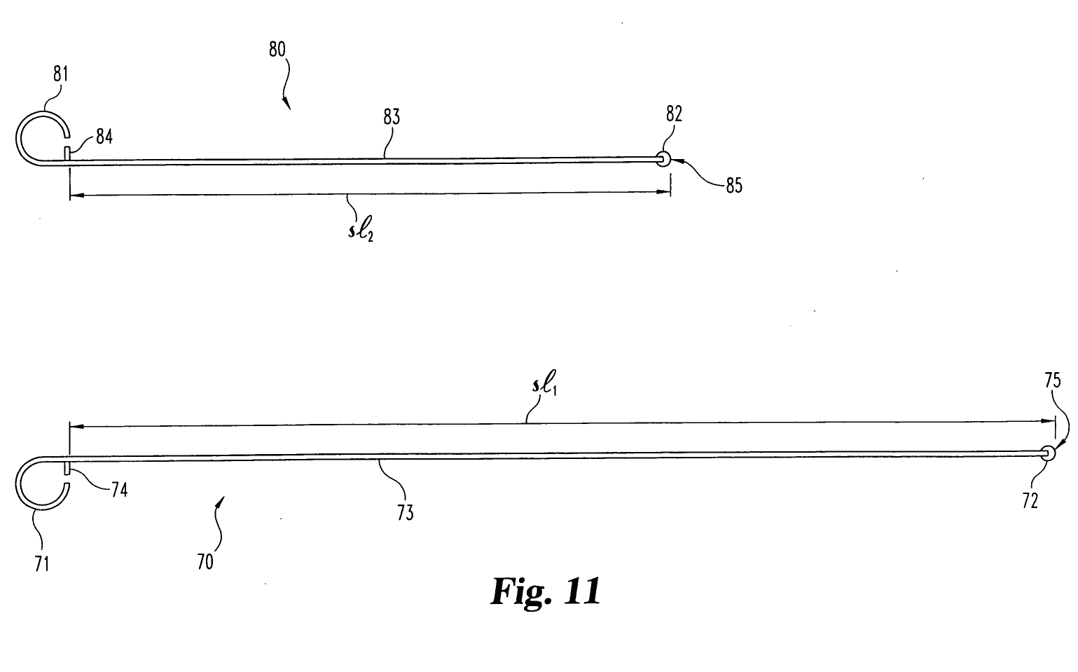

Further details regarding the stylets 70, 80 are illustrated in FIG. 11. With respect to stylet 70, it includes an elongated portion 73 extending between a proximal end 71 and a distal end 72. Stylet 80 includes an elongated portion 83 which extends between a proximal end 81 and a distal end 82. Each of the proximal ends 71, 81 is generally structured as a curved handle member to facilitate handling of stylets 70, 80. Moreover, each of the distal ends 72, 82 defines an enlarged head portion 75, 85 relative to the respective elongated portions 73, 83. Enlarged head portions 75, 85 are generally structured to be placed through the corresponding lumens of the catheter assembly 10 without puncturing or tearing any of the components of catheter assembly 10. Stylet 70 also includes a stop member 74 and a length sl₁ between stop member 74 and distal end 72. Stylet 80 also includes a stop member 84 and a length sl₂ between stop member 84 and distal end 82.

It should be appreciated that the length sl₁ is generally sized to be shorter than the overall length ol₁ of the members 30 and 50 between proximal end 52 and distal end 34, as illustrated in FIG. 10. Likewise, the length sl₂ is generally sized to be shorter than the overall length ol₂ of the members 40 and 50 between proximal end 52 and distal end 44, as also illustrated in FIG. 10. Accordingly, stylets 70, 80 can be inserted into catheter assembly 10 until the stop members 74, 84 contact the proximal end 52 of main member 50 with each of the enlarged head portions 75, 85 being positioned proximally of the distal ends 34, 44, respectively. Consequently, the enlarged head portions 75, 85 remain in the lumens of the respective intrabody members 30, 40 and are prevented from damaging anatomical features surrounding the implantation site of catheter assembly 10. It should be appreciated that stylets 70, 80 can also include additional non-illustrated features. For example, each might include depth markings on the elongated portion 73, 83 and/or may include an identification label so that a surgeon can readily ascertain lo the identity of the respective stylets 70, 80.

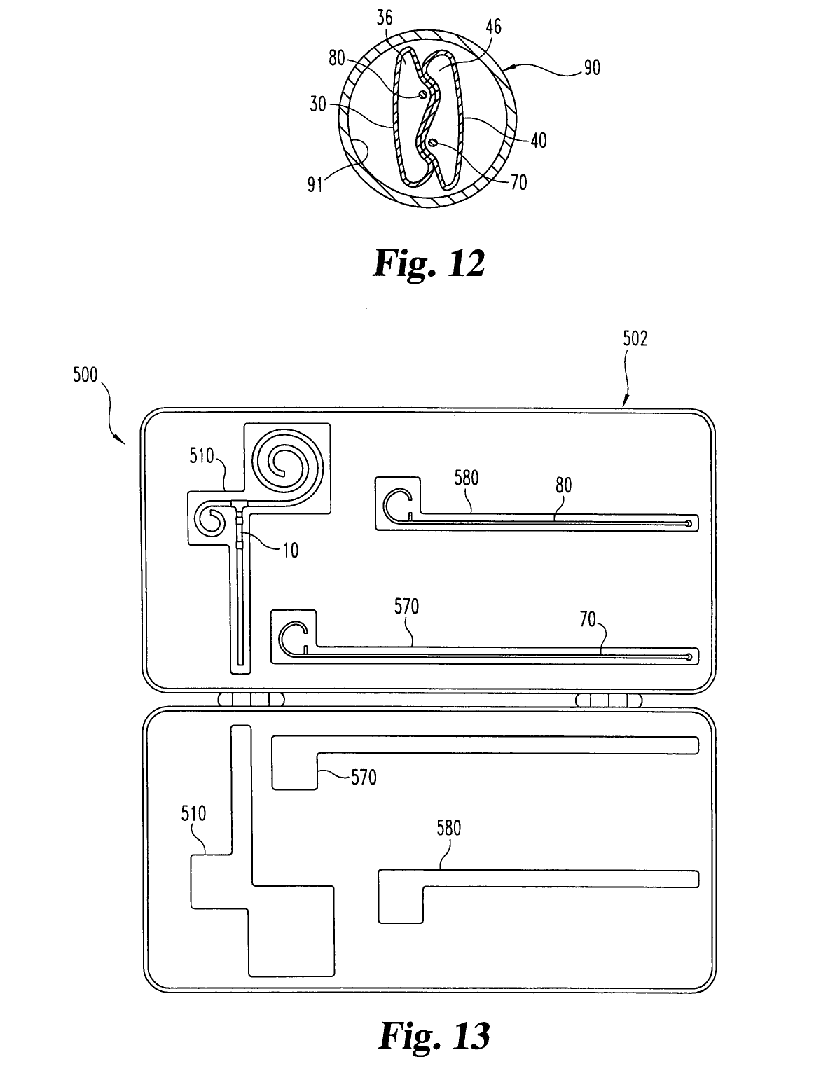

Once intrabody members 30, 40 have been straightened, catheter assembly 10 can be implanted at a surgical location. As illustrated in FIG. 12, a retractor sleeve 90 can aid in implantation of catheter assembly 10. Each of the intrabody members 30, 40 can be flattened from their circular shape into a substantially ovoid shape or other reduced profile configuration to facilitate placement thereof within the interior 91 of retractor sleeve 90. It should also be appreciated that the stylets 70, 80 can be placed substantially in axial alignment with one another along longitudinal axis L₂ in order to decrease the overall combined footprint of the intrabody members 30, 40 in the retractor sleeve 90 and further facilitate placement through interior 91 of retractor sleeve 90.

Catheter assembly 10 can be continually advanced through retractor sleeve 90 until intrabody members 30, 40 and coupling member 11 are positioned at a desired intrabody location. Once implanted, stylets 70, 80 can be withdrawn from catheter assembly 10 to allow intrabody members 30, 40 to reform the coiled sections 38, 48 toward their respective original configurations. In another procedure, it is contemplated that one or both of the stylets 70, 80 can be incrementally withdrawn from main member 50 as catheter assembly 10 is inserted through the retractor sleeve 90. Accordingly, one or both of the coiled sections 38, 48 can start reforming during the insertion of catheter assembly 10.

In one manner of implanting catheter assembly 10 in which members 30, 40 and 50 line in a single plane, first portion 12 of coupling member 11 is oriented such that axis L₁ lies along the patient's peritoneal membrane and second portion 13 of coupling member 11 is positioned such that axis L₂ lies at an angle less than 90° relative to the plane of the peritoneal membrane at the site of entry. In one embodiment, the angle between the plane of the peritoneal membrane and axis L₂ is an angle of from about 30° to about 60°. In another embodiment, the angle is an angle of about 45°. In this orientation, coiled portion 48 will be urged to lie along the peritoneal membrane in a plane parallel to the plane of the peritoneal membrane, and in the absence of external forces coiled portion 38 would be positioned in a plane at an angle to the peritoneal membrane corresponding to the angle of axis L₂. A similar orientation of coiled portions 38, 48 in the peritoneal cavity can be achieved by using a catheter assembly in which axis L₂ lies in a plane set at an angle relative to a the plane in which members 30 and 40 lie. Implantation of this catheter assembly embodiment such that axis L₂ lies substantially at a 90° angle with the plane of the peritoneal membrane will cause one of coiled section 38 and coiled section 48 to be urged against the peritoneal membrane as described above, depending up direction of the angle between axis L₂ and the plane in which members 30 and 40 lie.

In one or more forms, the methods contemplated herein can also include positioning one or more cuffs to promote tissue ingrowth. Additionally, while the insertion method described has made specific reference to catheter assembly 10, it should be appreciated that the method can also be readily applied to other catheter assembly embodiments discussed herein and encompassed by this application.

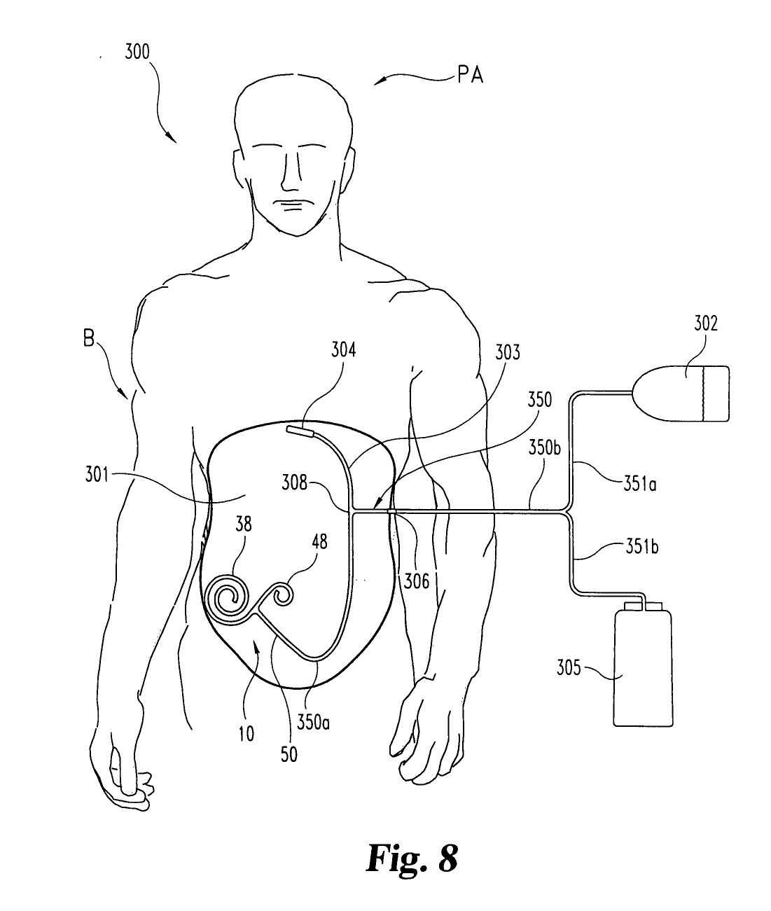

Further descriptions regarding alternative uses of implantable catheter assemblies according to the present application are set forth below in regard to FIGS. 7 and 8. More particularly, the systems 200, 300 illustrated in FIGS. 7 and 8 are used for different forms of Continuous Flow-through Peritoneal Dialysis. As one having skill in the art would appreciate, in Continuous Flow-through Peritoneal Dialysis a dialysate is supplied by a supply member to the peritoneal cavity while a spent dialysate is simultaneously drained from the peritoneal cavity by a drainage member, a process which consequently removes one or more toxins from a patient.

As illustrated in FIG. 7, for example, the system 200 includes a supply member 204 and an inferiorly positioned drainage member provided by catheter assembly 10 implanted in the peritoneal cavity 201 of the body B of patient PA. The supply member 204 is connected to a dialysate source 202 via a conduit 203 which enters the body B at a first position 206. The dialysate source 202 is generally structured to disperse a dialysate through conduit 203 and out of supply member 204 into the peritoneal cavity 201 of the patient PA. Moreover, main member 50 enters the body B at a second position 207 and is coupled with a waste container 205 positioned externally to the body B. In this form, the coiled sections 38, 48 of catheter assembly 10 can collect the dialysate as it flows through the peritoneal cavity and has removed toxins from the patient PA, or alternatively stated, has become “spent”. As the spent dialysate is collected by coiled sections 38, 48 it is distributed through the lumen of the main member 50 and into the waste container 205. In an alternate embodiment (not shown) each of conduit 203 and main member 50 can be connected to a single device that is effective to regenerate spent dialysate removed from the peritoneal cavity through main member 50 so that the regenerated dialysate can be reintroduced into the peritoneal cavity 201 through conduit 203. It should be appreciated that each of the supply conduit 203 and main member 50 can include a pair of spaced apart cuffs which are structured to promote tissue ingrowth at the abdominal wall and at or near the skin of the patient PA.

With respect to the system 300 illustrated in FIG. 8, system 30 operates in a manner similar to the system 200. However, unique to the system 300, the components thereof enter the body B at a single position 306 only. More particularly, the system 300 includes a supply member 304 and an inferiorly positioned drainage member in the form of catheter assembly 10 implanted in the peritoneal cavity 301 of the body B. Main member 50 is coupled with a single lumen portion 350a of conduit 350. Single lumen portion 350a extends from catheter assembly 10 to an intrabody coupling portion 308 to which the conduit 303 is also coupled. From coupling portion 308 to a position external to the body B, conduit 350 includes a dual lumen portion 350b which splits into separate conduits 351a and 351b which are coupled to a dialysate source 302 and a waste container 305, respectively (or to a single regeneration device (not shown) in other embodiments). Coupling portion 308 and dual lumen portion 350b can be partitioned similar to the embodiment illustrated in FIG. 9 which will be described in further detail below. In this form, a first lumen of the dual lumen portion 350b is in communication with conduit 351a and is able to supply a dialysate from source 302 to conduit 303 for distribution by supply member 304. Moreover, single lumen portion 350a directs spent dialysate collected by coiled sections 38, 48 to a second lumen of dual lumen portion 350b which is in communication with conduit 351b. Accordingly, the spent dialysate can be deposited in waste container 305 or in a regeneration device as described above.

With further reference to system 300, it should be appreciated that coupling portion 308 can be positioned in the peritoneal cavity 301 or some or all of coupling portion 308 alternatively can be positioned outside of the peritoneal cavity 301. As would be appreciated by one having skill in the art, the number of tissue promoting cuffs and the placement thereof in the system 300 can be determined by the positioning of coupling portion 308. For example, when coupling portion 308 is positioned in the peritoneal cavity 301, dual lumen portion 350b of transcutaneous member 350 may include a cuff positioned to promote tissue ingrowth at the abdominal wall and another cuff positioned to promote tissue ingrowth at or near position 306. However, when coupling portion 308 is positioned outside of the peritoneal cavity 301, dual lumen portion 350b may include a cuff positioned to promote tissue ingrowth at or near position 306 while each of the conduit 303 and the single lumen portion 350a may include a cuff positioned to promote tissue ingrowth at the abdominal wall.

While in the illustrated forms of the systems 200, 300 the coiled sections 38 have been positioned generally inferiorly of coiled sections 48, it should be appreciated that in alternative forms coiled sections 38, 48 can be in other positions relative to each other. For example, in one form the coiled sections 38 can be positioned superiorly of coiled sections 48 while in another form coiled sections 38, 48 can be superiorly/inferiorly aligned with each other. Furthermore, in one or more non-illustrated forms, it should be appreciated that one or both of the systems 200, 300 can include one or more pumps, filters, screens, valves, vacuums or additional componentry which may aid in the supply or drainage of dialysate or other fluid from the peritoneal cavity 201, 301.

Coupling portion 308 can have a configuration as illustrated in FIG. 9. In addition, coupling member 411 depicted in FIG. 9 can be used in place of coupling member 11 shown and described in connection with FIGS. 1 and 2 to provide an alternative embodiment catheter assembly 410 in which members 30 and 40 provide access to separate lumens of a dual lumen main member rather than access to a single lumen main member 50. While not shown in FIG. 9, it should be appreciated that each of the intrabody members 430, 440 can include a coiled section like coiled sections 38, 48. Similar to the coupling member 11, the proximal ends 432, 442 of the intrabody members 430, 440 are respectively coupled with access ports 414, 416 of coupling member 411. Coupling member 11 includes a partition 422 which separates the coupling member 411 into a first pathway 424 which is in fluid communication with lumen 436 of intrabody member 430 and a second pathway 426 which is in fluid communication with lumen 446 of intrabody member 440. Partition 422 also extends into main member 450 and divides it such that the first and second pathways 424, 426 also extend in main member 450. The partition 422 can extend in main member 450 to a position external to the patient, at which point the first and second pathways 424, 426 separate from one another into separate conduit members (not shown).

In this embodiment, it should be appreciated that one of the intrabody members 430, 440 can receive and disperse a fluid from one of the pathways 424, 426 while the other of the intrabody members 430, 440 can collect and direct a fluid from the patient into the other of the pathways 424, 426 to a position external to the patient. For example, in one procedure, catheter assembly 410 can be used for Continuous Flow-through Peritoneal Dialysis, the general function of which has been described herein. Likewise, second pathway 426 can supply a dialysate to lumen 446 of second member 440 where it is passed through openings (not shown) and into the peritoneal cavity of the patient. Moreover, first member 430 can collect the spent dialysate from the peritoneal cavity and pass it through lumen 432 and into first pathway 424 to a position external to the patient. In one manner of using assembly 410, the separate lumens can alternately be used to infuse dialysate and to withdraw spent dialysate from the peritoneal cavity. By periodically alternating the flow direction in members 30 and 40, it is possible that clearance can be enhanced over time. It should also be appreciated that pathways 424, 426 can both be used to supply the dialysate and then subsequently drain the dialysate once it has become spent.

The present application also contemplates a kit 500 for storing and transporting a catheter assembly which includes catheter assembly 10 and stylets 70, 80. Kit 500 includes a package 502 including internal compartments 510, 570 and 580 which are generally structured to hold catheter assembly 10 and stylets 70, 80. It should be appreciated that package 502 can sterilely seal and house catheter assembly 10 and stylets 70, 80 until use. Moreover, it should be appreciated that kit 500 can include one or more surgical aides, instruments and devices, including, for example, but not limited to, retractors, dilators, tissue promoting cuffs, sutures, needles, syringes, scalpels, scissors, forceps, hemostats, sponges, bandages, ointments, lubricating gels, antiseptics, antimicrobials, antibiotics, analgesics and instructions for use. Additionally, it should also be appreciated that one or more of the components of kit 500 may packaged or supplied individually.

While the invention has been illustrated and described in detail in the drawings and foregoing description, the same is to be considered illustrative and not restrictive in character, it being understood that only selected embodiments have been shown and described and that all changes, equivalents, and modifications that come within the scope of the inventions described herein or defined by the following claims are desired to be protected. Any experiments, experimental examples, or experimental results provided herein are intended to be illustrative of the present invention and should not be construed to limit or restrict the invention scope. Further, any theory, mechanism of operation, proof, or finding stated herein is meant to further enhance understanding of the present invention and is not intended to limit the present invention in any way to such theory, mechanism of operation, proof, or finding. In reading the claims, words such as “a”, “an”, “at least on”, and “at least a portion” are not intended to limit the claims to only one item unless specifically stated to the contrary. Further, when the language “at least a portion” and/or “a portion” is used, the claims may include a portion and/or the entire item unless specifically stated to the contrary.

What is claimed is:

1. A catheter assembly for use in peritoneal dialysis, comprising:
a first intraperitoneal member including a first coiled portion at a distal end thereof and defining a first lumen extending from a proximal end toward said distal end;
a second intraperitoneal member including a second coiled portion at a distal end thereof and defining a second lumen extending from a proximal end toward said distal end;
a coupling member including first and second access ports and a transabdominal member defining a third lumen; and
wherein said proximal end of said first intraperitoneal member extends from said coupling member at said first access port and said proximal end of said second intraperitoneal member extends from said coupling member at said second access port with said first and second lumens in fluid communication with said third lumen.

2. The assembly of claim 1, wherein said second coiled portion is more tightly coiled than said first coiled portion.

3. The assembly of claim 2, wherein said first intraperitoneal member further includes a first length between a proximal end and said distal end thereof and said second intraperitoneal member further includes a second length between a proximal end and said distal end thereof, said second length being smaller than said first length.

4. The assembly of claim 3, wherein said coupling member defines an internal chamber and said first, second and third lumens are in fluid communication with said internal chamber.

5. The assembly of claim 4, wherein said proximal ends of said first and second intraperitoneal members extend substantially along a common longitudinal axis.

6. The assembly of claim 5, wherein said first and second intraperitoneal members and said coupling member lie substantially in a common plane.

7. The assembly of claim 6, wherein said first coiled portion is positioned on a first side of said longitudinal axis and said second coiled portion is positioned on a substantially opposite second side of said longitudinal axis.

8. The assembly of claim 7, wherein said transabdominal member is positioned on said second side of said longitudinal axis.

9. The assembly of claim 8, wherein said first and second intraperitoneal members are straightened when in an implantation orientation.

10. The assembly of claim 9, wherein in said implantation orientation said coupling member is folded with said first and second access ports positioned adjacent one another.

11. The assembly of claim 10, wherein in said implantation orientation each of said first and second intraperitoneal members includes a substantially linear profile between said proximal and distal ends thereof.

12. The assembly of claim 3, further comprising a radiopaque marker disposed on an exterior surface of at least one of said first and second intraperitoneal members and said transabdominal member.

13. The assembly of claim 12, wherein said radiopaque marker is barium sulfate.

14. The assembly of claim 3, wherein each of said first and second lumens includes a substantially circular cross-sectional shape.

15. The assembly of claim 14, wherein each of said first and second lumens includes a diameter in the range from about 3 to about 5 millimeters.

16. The assembly of claim 14, wherein each of said first and second lumens is compressible to transition said circular shape to a flattened shape.

17. The assembly of claim 4, further comprising a partition in said internal chamber and said third lumen, said partition dividing said internal chamber and said third lumen into first and second pathways.

18. The assembly of claim 17, wherein said first pathway is in fluid communication with said first lumen and said second pathway is in fluid communication with said second lumen.

19. The assembly of claim 1, wherein each of said first and second intraperitoneal members includes a plurality of openings in communication with said lumen thereof.

20. The assembly of claim 19, wherein said plurality of openings includes a number of oppositely disposed openings spaced apart along respective ones of said first and second intraperitoneal members.

21. The assembly of claim 20, wherein a perimetrical position of said pairs of openings varies between adjacent ones of said pairs of openings.

22. The assembly of claim 1, further comprising a first tissue ingrowth promoting cuff positioned around said transabdominal member adjacent a distal end thereof.

23. The assembly of claim 22, further comprising a second tissue ingrowth promoting cuff positioned around said transabdominal member and spaced proximally from said first cuff.

24. The assembly of claim 1 wherein said first intraperitoneal member includes a first length between said coupling member and said distal end thereof and said second intraperitoneal member includes a second length between said coupling member and said distal end thereof which is shorter than said first length.

25. The assembly of claim 24, further comprising a transabdominal member extending from said coupling member.

26. The assembly of claim 24, wherein each of said first and second intraperitoneal members is structured to infuse a dialysate into the peritoneal cavity of a patient and drain a spent dialysate from the peritoneal cavity of the patient.

27. The assembly of claim 1 wherein:
said first coiled portion includes a first dimension corresponding to an outer diameter thereof;
said second coiled portion includes a second dimension corresponding to an outer diameter thereof; and
said second dimension is smaller than said first dimension.

28. The assembly of claim 27, wherein said first and second intraperitoneal members and said transabdominal member lie substantially in a common plane.

29. The assembly of claim 28, wherein said transabdominal member extends along a first longitudinal axis and each of said first and second intraperitoneal members are positioned on opposite sides of said longitudinal axis.

30. The assembly of claim 29, wherein said coupling member includes a first portion from which said first and second intraperitoneal members extend, said first portion extending along a second longitudinal axis positioned substantially transversely to said first longitudinal axis.

31. The assembly of claim 30, wherein each of said first and second coiled portions are positioned substantially on opposite sides of said second longitudinal axis.

32. A catheter assembly, comprising:
a resilient, pliable coupling member defining an internal chamber, said coupling member including a first portion with a first access port and a second access port and a second portion defining a third access port, said first, second and third access ports being in communication with said internal chamber;
a first intrabody member extending from said first access port and a second intrabody member extending from said second access port, wherein each of said first and second intrabody members defines a lumen in communication with said internal chamber; and
wherein the catheter assembly is configurable to an implantation orientation wherein each of said first and second intrabody members is in a straightened configuration and an implanted orientation wherein each of said first and second intrabody members is in a contracted configuration.

33. The assembly of claim 32, further comprising a main member extending from said third access port along a first longitudinal axis, said main member defining a lumen in communication with said internal chamber.

34. The assembly of claim 33, wherein said coupling member is foldable between said implantation orientation and said implanted orientation.

35. The assembly of claim 34, wherein in said implantation orientation said first and second access ports are juxtaposed and in said implanted orientation said first and second access ports lie along a second longitudinal axis transversely oriented to said first longitudinal axis.

36. The assembly of claim 33, wherein in said contracted configuration each of said first and second intrabody members includes a coiled portion.

37. The assembly of claim 36, wherein in said implanted orientation said first portion of said coupling member extends along a second longitudinal axis and said coiled portion of said first intrabody member is positioned on a first side of said second longitudinal axis and said coiled portion of said second intrabody member is positioned on an opposite side of said second longitudinal axis.

38. The assembly of claim 37, wherein in said implanted orientation said first and second intrabody members, said coupling member and said main member are positioned substantially in a common plane.

39. The assembly of claim 36, wherein said coiled portion of said second intrabody member is more tightly coiled than said coiled portion of said first intrabody member.

40. The assembly of claim 39, wherein each of said first and second intrabody members includes a distal end spaced apart from respective ones of said first and second access ports and wherein in said contracted configuration said distal end is positioned adjacent a center of said coiled portion of said corresponding intrabody member.

41. The assembly of claim 40, wherein said first intrabody member has a length between said first access port and said distal end which is greater than a length of said second intrabody member between said second access port and said distal end thereof.

42. The assembly of claim 33, further comprising a radiopaque marker disposed on an exterior surface of at least one of said first and second intrabody members and said main member.

43. The assembly of claim 42, wherein said radiopaque marker is barium sulfate.

44. The assembly of claim 33, wherein said lumens of said first and second intrabody members define a substantially circular cross-section.

45. The assembly of claim 44, wherein said lumens of said first and second intrabody members include a diameter in the range from about 3 to about 5 millimeters.

46. The assembly of claim 45, wherein said diameter of said lumens of said first and second intrabody members is about 4 millimeters.

47. The assembly of claim 44, wherein each of said first and second intrabody members includes a plurality of openings in communication with said lumen thereof.

48. The assembly of claim 47, wherein said plurality of openings includes a number of pairs of oppositely disposed openings spaced apart along respective ones of said first and second intrabody members.

49. The assembly of claim 48, wherein adjacent ones of said pairs of opposite openings open at circumferentially offset locations.

50. The assembly of claim 33, further comprising a first tissue ingrowth promoting cuff positioned around said main member adjacent to said third access port.

51. The assembly of claim 50, further comprising a second tissue ingrowth promoting cuff positioned around said main member and spaced apart from said first cuff.

52. The assembly of claim 33, further comprising a partition positioned in said internal chamber and in said lumen of said main member, said partition dividing said internal chamber and said lumen of said main member into first and second pathways.

53. The assembly of claim 52, wherein said first pathway is in fluid communication with said lumen of said first intrabody member and said second pathway is in fluid communication with said lumen of said second intrabody member.

54. A catheter assembly comprising:
an elongated main member including a coupling portion disposed at a distal end thereof;
a first intrabody member including a proximal portion and a distal portion, said distal portion defining a first coiled section;
a second intrabody member including a proximal portion and a distal portion, said distal portion defining a second coiled section; and
wherein said proximal portions of said first and second intrabody members extend from said coupling portion along a first longitudinal axis and said first and second coiled sections are positioned on substantially opposite sides of said first longitudinal axis.

55. The catheter assembly of claim 54, wherein said main member is positioned on the same side of said longitudinal axis as said second coiled section.

56. The assembly of claim 54, wherein each of said first and second intrabody members and said main member defines a lumen in communication with an internal chamber defined by said coupling portion.

57. The assembly of claim 56, wherein each of said lumens of said first and second intrabody members includes a plurality of openings in fluid communication therewith.

58. The assembly of claim 56, further comprising a partition in said coupling portion and said lumen of said main member, said partition dividing said coupling portion and said lumen of said main member into a first pathway in fluid communication with said lumen of said first intrabody member and a second pathway in fluid communication with said lumen of said second intrabody member.

59. The assembly of claim 57, wherein said plurality of openings is structured to dispense a liquid from said lumens into a patient.

60. The assembly of claim 59, wherein said plurality of openings is further structured to drain a liquid from the patient.

61. The assembly of claim 54, wherein said first intrabody member includes a first length between a proximal end and a distal end thereof that is greater than a second length of said second intrabody member between a proximal and a distal end thereof.

62. The assembly of claim 54, wherein said main member extends along a second longitudinal axis oriented transversely to said first longitudinal axis.

63. The assembly of claim 54, wherein said second coiled section is more tightly coiled than said first coiled section.

64. The assembly of claim 54, wherein said main member includes a first tissue ingrowth promoting cuff positioned adjacent to said distal end thereof and a second tissue ingrowth promoting cuff spaced proximally from said first cuff.

65. A system for performing peritoneal dialysis, comprising;
an intraperitoneal supply member structured to infuse the peritoneal cavity with a dialysate; and
an intraperitoneal drain member spaced from said supply member, said drain member including a first collecting member including a first coiled section and a second collecting member including a second coiled section, said collecting members being structured to remove said dialysate from the peritoneal cavity.

66. The system of claim 65, further comprising a drainage conduit coupled with said drain member, said drainage conduit being structured to deliver said dialysate from said first and second collecting members to a location external to a patient.

67. The system of claim 66, wherein said drainage conduit is sized to extend beyond the epidermis of a patient.

68. The system of claim 67, wherein said drainage conduit includes a first tissue ingrowth promoting cuff and a second tissue ingrowth promoting cuff spaced apart from said first cuff.

69. The system of claim 65, further comprising a transcutaneous member including an intrabody coupling portion defining a first access port and a second access port, said transcutaneous member and said coupling portion being partitioned into first and second pathways and wherein said first pathway is in fluid communication with said first access port to which said supply member is coupled and said second pathway is in fluid communication with said second access port to which a drainage conduit extending from said drainage member is coupled.

70. The system of claim 69, wherein said transcutaneous member includes a first tissue ingrowth promoting cuff positioned adjacent to said intrabody coupling portion and a second tissue ingrowth promoting cuff spaced apart from said first cuff.

71. The system of claim 70, wherein said transcutaneous member is divided into first and second conduit members at a position external to the body and said first conduit member is coupled with a dialysate source and said second conduit member is coupled to a receptacle for spent dialysate.

72. The system of claim 65, wherein said first collecting member includes a first length between a proximal end and a distal end thereof and said second collecting member includes a second length between a proximal end and a distal end thereof which is shorter than said first length.

73. The system of claim 72, wherein said first collecting member and said second collecting member extend in opposite directions from one another.

74. A method for positioning a catheter assembly for peritoneal dialysis, comprising:
providing a catheter assembly including a coupling member, said coupling member including a transabdominal member and first and second intraperitoneal members extending therefrom, said first and second intraperitoneal members including distal coiled sections;
providing a pair of stylets configured to pass through said coupling member and into communication with lumens of said first and second intraperitoneal members;
inserting a first one of said stylets into said lumen of said first intraperitoneal member and thereby substantially straightening said distal coiled section thereof;
inserting a second one of said stylets into said lumen of said second intraperitoneal member and thereby substantially straightening said distal coiled section thereof;
implanting said catheter assembly at a prepared surgical location with said coiled sections substantially straightened; and
removing said first and second stylets from said lumens of said intraperitoneal members and thereby reforming said distal coiled sections to a coiled configuration.

75. The method of claim 74, further comprising folding said coupling member and wherein inserting said stylets includes inserting said first and second stylets through said transabdominal member and into said lumens.

76. The method of claim 74, wherein said coiled section of said second intraperitoneal member is more tightly coiled than said coiled section of said first intraperitoneal member.

77. The method of claim 76, wherein each of said first and second intraperitoneal members has a length between a proximal and a distal end and wherein said length of said first intraperitoneal member is greater than said length of said second intraperitoneal member.

78. The method of claim 74, wherein said catheter assembly includes a radiopaque marker on an exterior portion thereof and said implanting includes radiographically monitoring the position of said radiopaque marker.

79. The method of claim 74, wherein said transabdominal member includes a first cuff portion and a second cuff portion spaced apart from said first cuff portion.

80. The method of claim 79, further comprising positioning said first and second cuffs at said surgical site to promote tissue ingrowth between said first and second cuffs and around said transabdominal member.

81. The method of claim 74, wherein removing said first and second stylets includes incrementally withdrawing said first and second stylets from said lumens of said first and second intraperitoneal members to gradually reform said coiled sections.

82. The method of claim 74, wherein implanting said catheter assembly includes passing said catheter assembly through a retractor sleeve.

83. The method of claim 82, wherein passing said catheter assembly further includes compressing the lumens of said first and second intraperitoneal members and positioning said stylets in a side-by-side relationship.

84. The method of claim 82, wherein implanting said catheter assembly further comprises positioning said first intraperitoneal member inferiorly of said second intraperitoneal member at the surgical location.

85. The method of claim 72, wherein inserting said first and second stylets includes inserting said stylets into said transabdominal member until a stop on each of said stylets contacts a proximal end of said transabdominal member.

86. The method of claim 83, wherein said first stylet includes a length between a distal end and said stop thereof which is greater than a length of said second stylet between a distal end and said stop thereof.

87. A kit for implanting a catheter assembly, comprising:
a catheter assembly including a main member and first and second intrabody members extending from a coupling member, said first and second intrabody members each including a coiled portion positioned at a distal end thereof;
a first stylet and a second stylet, wherein each of said first and second stylets includes an elongated shaft portion and an enlarged head portion at a distal end of said shaft portion; and
packaging for holding said catheter assembly and said first and second stylets in a sterilized condition until implantation of said catheter assembly.

88. The kit of claim 87, wherein each of said first and second intrabody members and said main member defines a lumen and said enlarged head portion of said first and second stylets is structured to pass through said lumen of each of said first and second intrabody members and said main member.

89. The kit of claim 88, wherein each of said first and second stylets further includes a stop portion spaced proximally from said enlarged head portion.

90. The kit of claim 89, wherein said first stylet includes a length between said enlarged head portion and said stop portion thereof which is greater than a length of said second stylet between said enlarged head portion and said stop portion thereof.

91. The kit of claim 90, wherein said length of said first stylet corresponds to a combined length of said first intrabody member and said main member to control an insertion distance of said first stylet into said first intrabody member.

92. The kit of claim 90, wherein said length of said second stylet corresponds to a combined length of said second intrabody member and said main member to control an insertion distance of said second stylet into said second intrabody member.

93. The kit of claim 87, further comprising a surgical aide selected from the group consisting of: retractors, dilators, tissue promoting cuffs, sutures, needles, syringes, scalpels, scissors, forceps, hemostats, sponges, bandages, ointments, lubricating gels, antiseptics, antimicrobials, antibiotics, analgesics and instructions for implanting said catheter assembly.
